(19) United States
(12) Patent Application Publication
(10) Pub. No.: US 2022/0183601 A1
(43) Pub. Date: Jun. 16, 2022

(54) MINIATURE SAFETY BLOOD LANCET FOR MINIMIZING PAIN

(71) Applicant: Tianjin Huashang Medical Equipment Co., Ltd., Tianjin (CN)

(72) Inventors: Jiazhao HE, Shijiazhuang (CN); Yanhong FENG, Chengde (CN); Aihui WANG, Tianjin (CN)

(73) Assignee: Tianjin Huashang Medical Equipment Co., Ltd., Tianjin (CN)

(21) Appl. No.: 17/313,009

(22) Filed: May 6, 2021

(30) Foreign Application Priority Data
Dec. 11, 2020 (CN) 202011458920.X

(51) Int. Cl. A61B 5/15; A61B 5/151

(52) U.S. Cl. CPC A61B 5/150412; A61B 5/1513; A61B 5/15117; A61B 5/150595

(57) ABSTRACT

A miniature safety blood lancet for minimizing pain includes a needle body, an activation sleeve, a housing and a spring. The activation sleeve is arranged in the housing. The needle body is arranged in the activation sleeve. The housing, the activation sleeve and the needle body are slidably connected. The activation sleeve is provided with an inner wall. The inner side of the middle of the inner wall is provided with an unlocking arm, and the outer side of the bottom of the inner wall is provided with a buckle. A needle cap is arranged at the front end of the needle body. The unlocking arm of the activation sleeve is snap-fitted with a self-locking protrusion of the needle cap and an inverted buckle of the housing, respectively. The spring is arranged between the bottom of the needle body and the housing.

### CROSS REFERENCE TO THE RELATED APPLICATIONS

This application is based upon and claims priority to Chinese Patent Application No. 202011458920.X, filed on Dec. 11, 2020, the entire contents of which are incorporated herein by reference.

### TECHNICAL FIELD

The present invention relates to the technical field of medical supplies, and more particularly, to a miniature safety blood lancet for minimizing pain.

### BACKGROUND

To use a typical blood lancet, the activation sleeve is generally pressed so that the needle body rotates to the launching position. In this case, the activation sleeve only moves axially within the housing of the blood lancet. However, since one end of the spring is connected to the needle body and the other end of the spring is connected to the bottom cap of the blood lancet without relative displacement, the rotation of the needle body will drive the spring to twist. As a result, the spring bounces while twisting, which impairs its potential energy, thereby affecting its bouncing force. Moreover, it is necessary to prevent the blood lancet from being unintentionally launched. In this regard, a cap device is arranged on the blood lancet to cover the inner sleeve that is capable of triggering a blood sampling operation. A cap device available on the market includes an excess of two components and thus has a complex structure, which not only increases its production cost but also causes waste of resources.

Additionally, the penetration depth of the blood lancet typically has some errors as it is controlled by the spring capable of being freely stretched and compressed, which may increase the pain. In the process of assembling and transporting the blood lancet, the cap is prone to be crooked, causing the needle tip and the needle body to tilt or bend, thereby increasing the pain of the user.

### SUMMARY

An objective of the present invention is to provide a miniature safety blood lancet for minimizing pain.

To achieve the above-mentioned objective, the present invention employs the following technical solutions.

A miniature safety blood lancet for minimizing pain includes: a needle body, an activation sleeve, a housing and a spring. The activation sleeve is arranged in the housing. The needle body is arranged in the activation sleeve. The housing, the activation sleeve and the needle body are slidably connected. The housing is provided with an inverted buckle. The activation sleeve is provided with an inner wall. The inner side of the middle of the inner wall is provided with an unlocking arm. The outer side of the bottom of the inner wall is provided with a buckle. A needle cap is arranged at the front end of the needle body. The needle cap is provided with a self-locking protrusion. The unlocking arm of the activation sleeve is snap-fitted with the self-locking protrusion of the needle cap and the inverted buckle of the housing, respectively, to fix the positional relationship between the needle body, the activation sleeve and the housing. The spring is arranged between the bottom of the needle body and the housing. A guide table is arranged at the bottom of the housing. The activation sleeve is provided with a bevel. The top of the guide table abuts the inner side of the bevel. When the activation sleeve is pressed, the top of the guide table expands the inner wall towards both sides, so that the needle body moves axially to pass through a clamping position of the inner wall under the force of the spring.

Preferably, both sides of the needle body are provided with limiting steps and latching steps. The rear of the activation sleeve is provided with a guide port matched with the limiting steps and the latching steps. A guide groove matched with the limiting steps and the latching steps is arranged within the housing. The limiting steps, the latching steps, the guide port, and the guide groove are located in the same direction.

Preferably, the unlocking arm is provided with an upper plane matched with the limiting steps, and the upper plane of the unlocking arm restricts the needle body from continuing to move axially to control the penetration depth of the needle body.

Preferably, a slide groove is arranged within the housing, and the outer side of the inner wall is provided with a guide rail matched with the slide groove.

Preferably, a needle core is pre-embedded in the needle body.

Preferably, the guide table is arranged on the bottom side wall of the housing. The guide table abuts both sides of the spring to guide the direction in which the spring is stretched and compressed and restrict a lateral movement of the spring.

Preferably, a truncated cone is arranged at the bottom of the housing. One end of the spring is sleeved on the bottom of the needle body and passes over a protrusion.

The other end of the spring is sleeved on the truncated cone of the housing.

Preferably, the miniature safety blood lancet for minimizing pain further includes a protective cap. The protective cap is sleeved on the needle cap, and is snap-fitted with the needle cap.

The present invention has the following advantages.

1) The penetration depth of the needle core of the needle body is controlled by the limiting steps of the needle body matched with the plane of the activation sleeve, thereby precisely positioning the penetration depth to reduce the pain during blood sampling.

2) The needle cap is fitted with the inner wall of the activation sleeve to prevent the cap from being crooked without introducing extra components, thereby eliminating the assembly process and reducing the cost.

3) The self-locking protrusion of the needle cap is fitted with the unlocking arm of the activation sleeve to prevent unintentional activation. The need body has a simple structure to reduce unnecessary waste of resources and is prevented from being unintentionally activated.

4) When the protective cap is in use, the needle cap can be separated from the needle body conveniently by rotating the protective cap, which further prevents the cap from being crooked and prevents unintentional activation.

### BRIEF DESCRIPTION OF THE DRAWINGS

The specific embodiments of the present invention will be described in more detail below with reference to the drawings.

FIG. 1 is a perspective view of the overall structure of a miniature safety blood lancet for minimizing pain according to an embodiment of the present invention;

FIG. 2 is a front view of the overall structure of the miniature safety blood lancet for minimizing pain according to the embodiment of the present invention;

FIG. 3 is a cross-sectional view taken along arrow F-F of FIG. 2;

FIG. 4 is a side view of the overall structure of the miniature safety blood lancet for minimizing pain according to the embodiment of the present invention;

FIG. 5 is a cross-sectional view taken along arrow J-J of FIG. 4;

FIG. 6 is a perspective view of the structure of a needle body of a miniature safety blood lancet for minimizing pain according to an embodiment of the present invention;

FIG. 7 is a front view of the structure of the needle body of the miniature safety blood lancet for minimizing pain according to the embodiment of the present invention;

FIG. 8 is a perspective view of the structure of an activation sleeve of the miniature safety blood lancet for minimizing pain according to the embodiment of the present invention;

FIG. 9 is a side view of the structure of the activation sleeve of the miniature safety blood lancet for minimizing pain according to the embodiment of the present invention;

FIG. 10 is a cross-sectional view taken along arrow F-F of FIG. 9;

FIG. 11 is a front view of the structure of the activation sleeve of the miniature safety blood lancet for minimizing pain according to the embodiment of the present invention;

FIG. 12 is a cross-sectional view taken along arrow G-G of FIG. 11;

FIG. 13 is a side view of the structure of a housing of the miniature safety blood lancet for minimizing pain according to the embodiment of the present invention;

FIG. 14 is a cross-sectional view taken along arrow G-G of FIG. 13;

FIG. 15 is a front view of the structure of the housing of the miniature safety blood lancet for minimizing pain according to the embodiment of the present invention;

FIG. 16 is a cross-sectional view taken along arrow F-F of FIG. 15;

FIG. 17 is a top view of the structure of the housing of the miniature safety blood lancet for minimizing pain according to the embodiment of the present invention;

FIG. 18 is a perspective view of the structure of the housing of the miniature safety blood lancet for minimizing pain according to the embodiment of the present invention;

FIG. 19 is a perspective view of the structure of a protective cap of the miniature safety blood lancet for minimizing pain according to the embodiment of the present invention;

FIG. 20 is a top view of the structure of the protective cap of the miniature safety blood lancet for minimizing pain according to the embodiment of the present invention;

FIG. 21 is a front view of the structure of the protective cap of the miniature safety blood lancet for minimizing pain according to the embodiment of the present invention;

FIG. 22 is a cross-sectional view taken along arrow H-H of FIG. 21;

FIG. 23 is a schematic view of the overall structure of a miniature safety blood lancet for minimizing pain according to embodiment 2;

FIG. 24 is a schematic view of the structure of a needle body of the miniature safety blood lancet for minimizing pain according to embodiment 2.

In the figures:

1. needle body; 11. needle cap; 12. self-locking protrusion; 13. limiting step; 14. latching step; 15. fixing buckle; 16. through hole; 17. tail part of the needle body; 18. protrusion; 2. activation sleeve; 21. inner wall; 22. unlocking arm; 23. inner hole; 24. guide port; 25. clamping position; 26. buckle; 27. guide rail; 28. bevel; 3. housing; 31. guide groove; 32. slide groove; 33. inverted buckle; 34. groove; 35. truncated cone; 36. guide table; 37. transition surface; 4. needle core; 5. spring; 6. protective cap; 61. inner circular hole; 62. inner flat hole; and 63. through hole.

### DETAILED DESCRIPTION OF THE EMBODIMENTS

For illustrating the present invention more clearly, the present invention will be further described in combination with the preferred embodiments. It should be understood by those skilled in the art that the details described below are illustrative rather than restrictive and shall not be construed as limiting the scope of protection of the present invention.

In the description of the present invention, it should be understood that the orientation or positional relations indicated by the terms such as “central”, “upper”, “lower”, “front”, “rear”, “left”, “right”, “vertical”, “horizontal”, “top”, “bottom”, “inner/inside”, and “outer/outside” are the orientation or positional relations as shown in the drawings only for ease of describing the present invention and simplifying the description, rather than indicating or implying that the indicated devices or elements must have the particular orientation, or be constructed and operated in that particular orientation, and thus cannot be construed as a limitation to the present invention.

In the description of the present invention, it should be noted that, unless otherwise expressly specified and limited, the terms such as “install/mount”, “connected to” and “connection” should be understood in a broad sense. For example, a connection may be a fixed, or may be a detachable connection, or may be an integral connection; it may be a direct connection, or may be an indirect connection through an intermediate, or may be an internal communication between two elements. For those of ordinary skill in the art, the specific meanings of the above terms in the present invention can be understood according to the specific situations.

### Embodiment 1

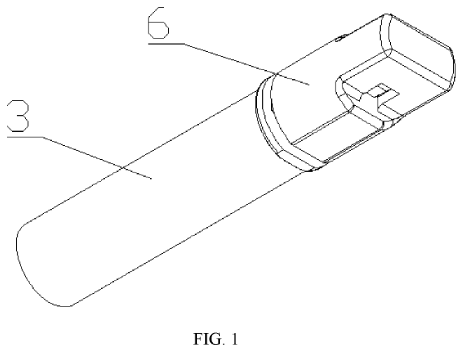

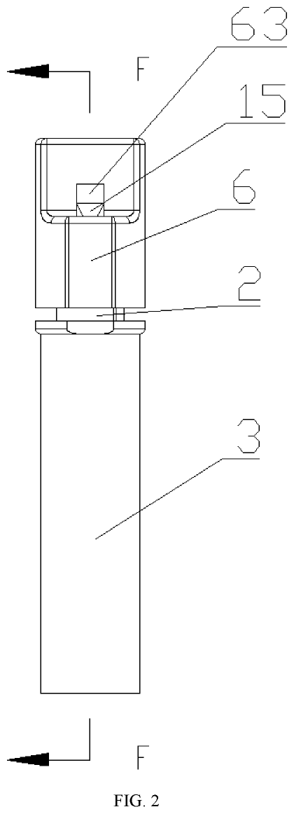

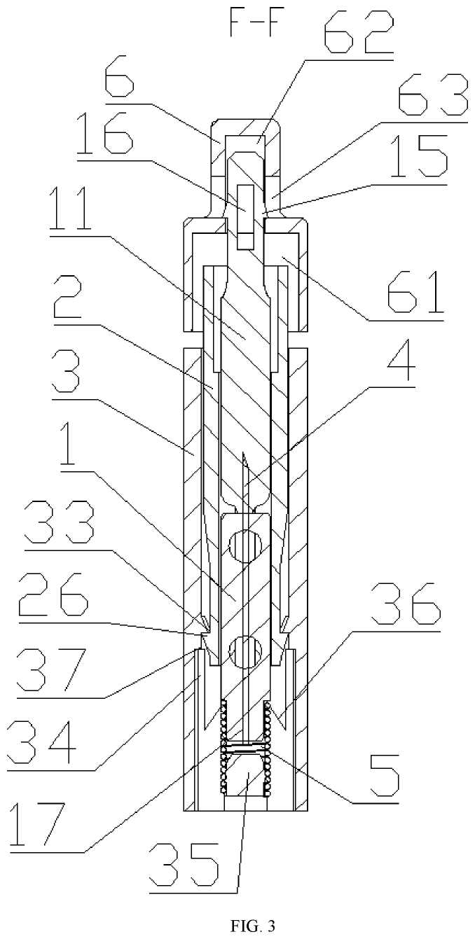

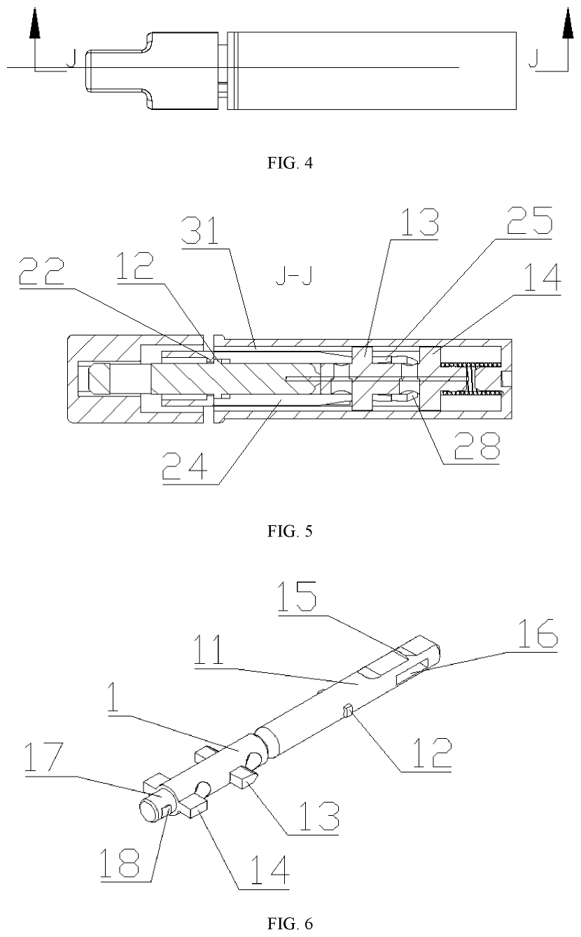

As shown in FIGS. 1 to 5, a miniature safety blood lancet for minimizing pain according to the present invention includes the needle body 1, the activation sleeve 2, the housing 3, the spring 5 and the protective cap 6. The needle body 1, the activation sleeve 2 and the spring 5 are arranged in the housing 3. One end of the spring 5 is mounted at the tail part 17 of the needle body, and the other end of the spring 5 is mounted at the bottom of the housing 3.

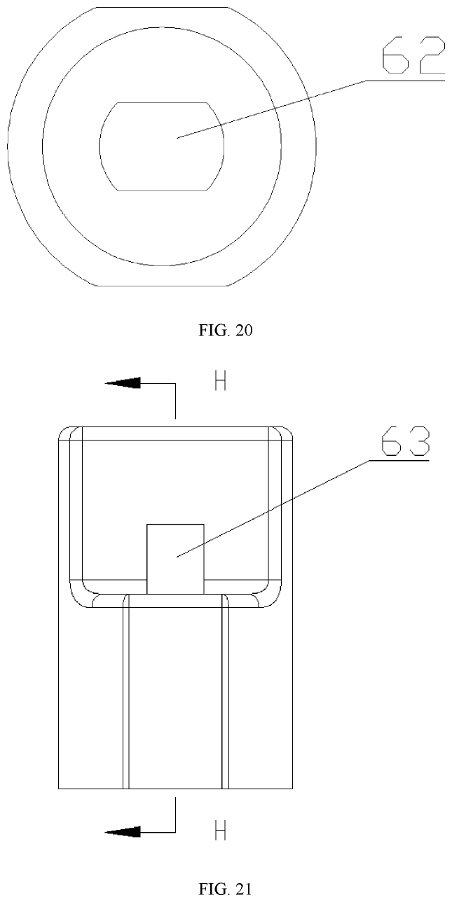

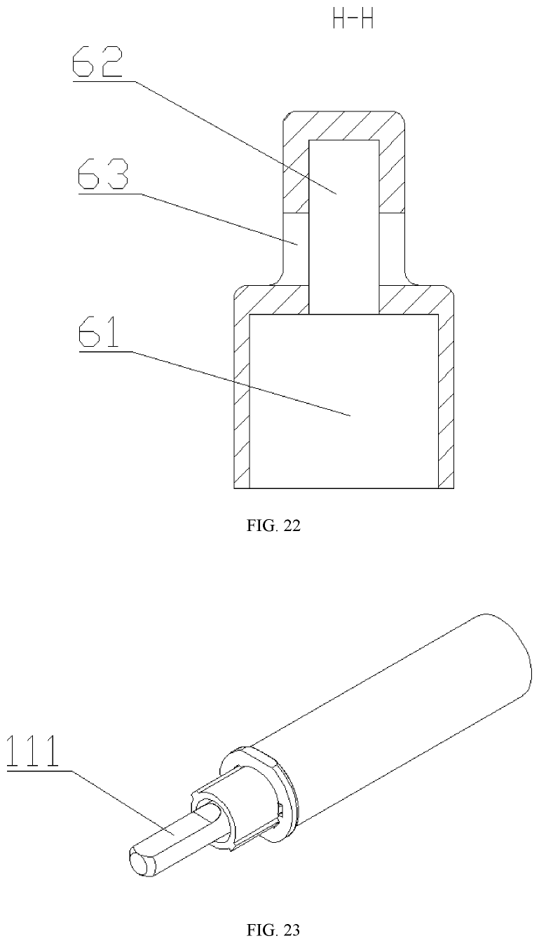

As shown in FIGS. 19 to 22, the front end of the protective cap 6 is provided with the inner circular hole 61. The rear end of the protective cap 6 is provided with the inner flat hole 62. The inner flat hole 62 is provided with the through hole 63.

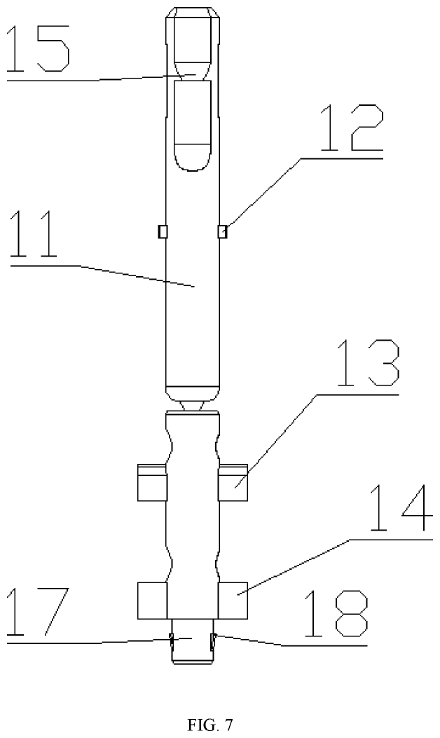

As shown in FIGS. 3, 6, and 7, the front end of the needle body 1 is provided with the needle cap 11. The needle cap 11 is provided with the through hole 16. Each of both sides of the through hole 16 is provided with the fixing buckle 15. The fixing buckle 15 is snap-fitted within the through hole 63 of the protective cap 6, so that the protective cap 6 and the needle cap 11 are assembled together. In use, the protective cap 6 is rotated to separate the needle cap 11 from the needle body 1.

The needle cap 11 is provided with the self-locking protrusion 12. Both sides of the needle body 1 are provided with the limiting steps 13 and the latching steps 14. The rear end of the needle body 1 is provided with the tail part 17. The protrusion 18 is arranged on the bottom circumferential wall of the needle body 1.

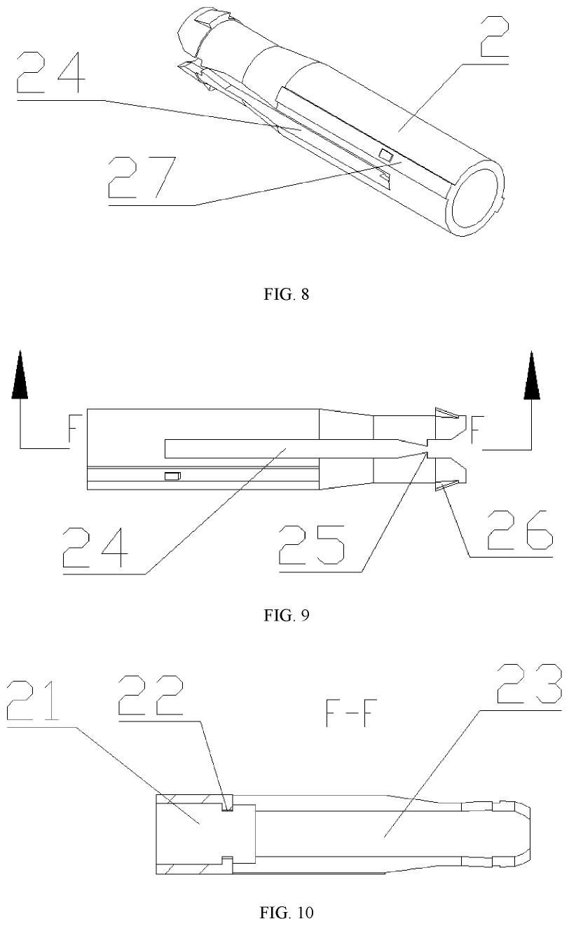

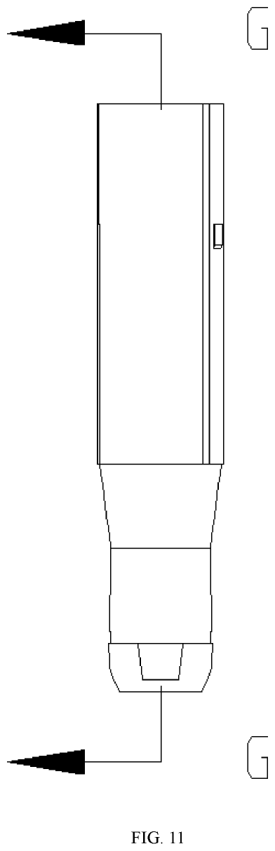

As shown in FIGS. 8 to 12, each of both sides of the outer side of the activation sleeve 2 is provided with the guide rail 27. The bottom of the outer side of the activation sleeve 2 is provided with the buckle 26. The activation sleeve 2 is internally provided with the inner wall 21. The inner wall 21 is provided with the unlocking arm 22. The unlocking arm 22 is provided with the guide port 24. Each of both sides of the guide port 24 is provided with the clamping position 25. Each of both sides of the tail part of the guide port 24 is provided with the bevel 28. The inner wall 21 is provided with the inner hole 23.

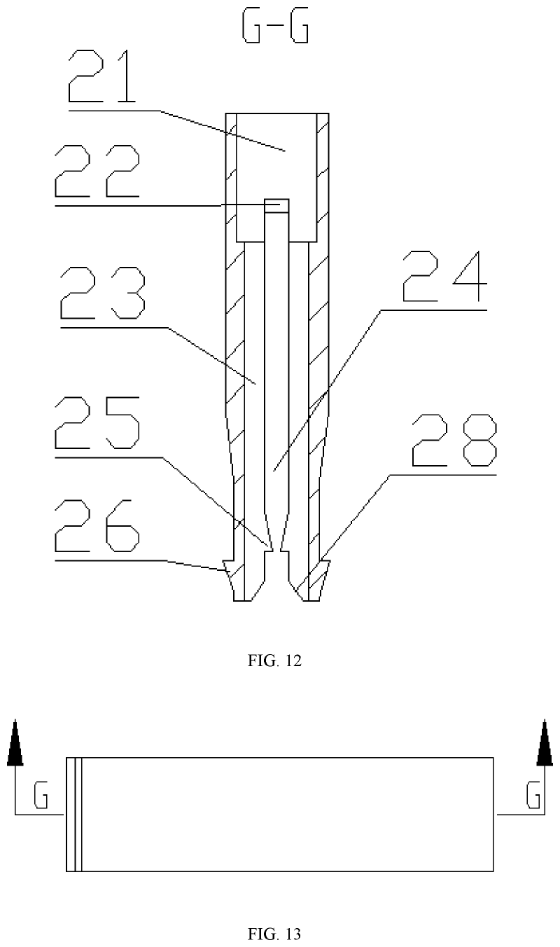

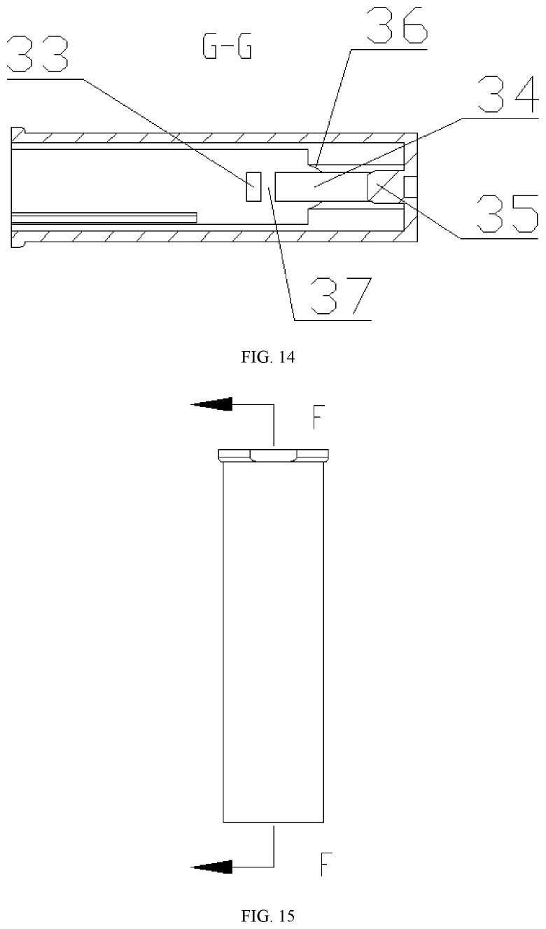

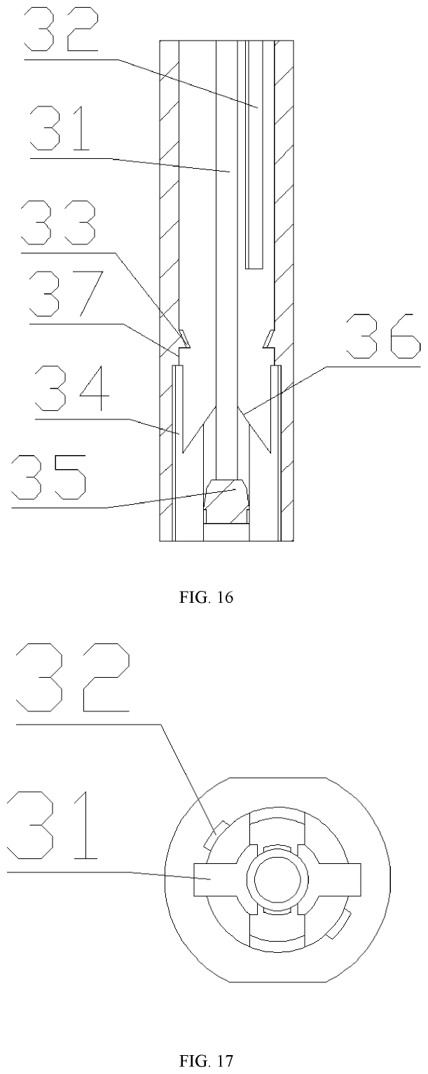

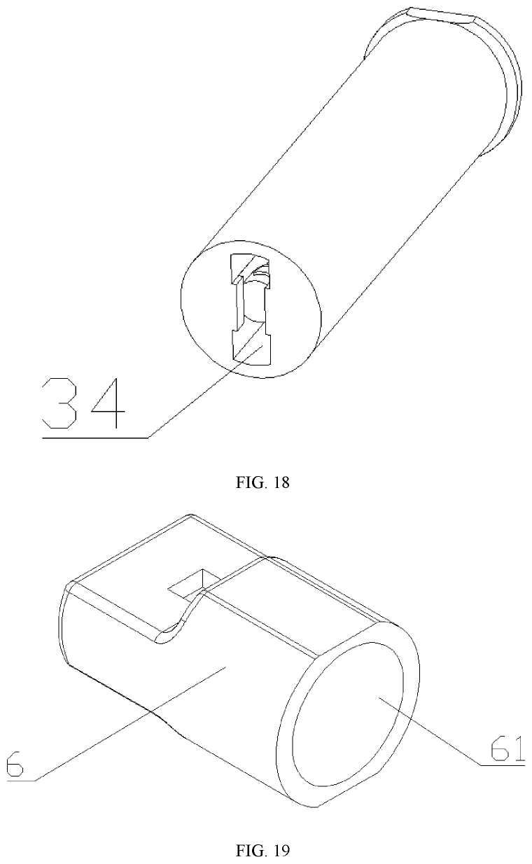

As shown in FIGS. 13 to 18, the front end of the housing 3 is provided with an opening. Each of both sides of the inner wall at the front end of the housing 3 is provided with the guide groove 31. The slide groove 32 is beside the guide groove 31. Each of both sides of the inside of the housing 3 is provided with the inverted buckle 33. The inverted buckle 33 is provided with the transition surface 37. The transition surface 37 is provided with the groove 34. Each of both sides of the bottom of the inside of the housing 3 is provided with the guide table 36. The truncated cone 35 is arranged at the bottom of the housing 3.

As shown in FIGS. 3 and 5, the buckle 26 at the tail part of the activation sleeve is located above the inverted buckle 33 on the inner side of the housing. The upper part of the buckle 26 is provided with the transition surface 37. The upper part of the transition surface 37 is provided with the groove 34. Each of both sides of the activation sleeve is provided with the guide port 24. The tail part of the guide port 24 is provided with the clamping position 25. The latching steps 14 of the needle body are located above the clamping position 25. The activation sleeve 2 and the housing 3 jointly constitute a structure for triggering the needle body 1 based on a principle as follows. The needle body 1 is slidably fitted with the activation sleeve 2. When the needle body 1 is mounted into the activation sleeve 2, the needle body 1 restricts the activation sleeve 2 from contracting inward. In this case, the outer side of the buckle 26 at the tail part of the activation sleeve is slidably fitted with the transition surface 37 on the inner side of the housing to prevent the activation sleeve 2 from expanding outward. In addition, the outer side of the buckle 26 at the tail part of the activation sleeve is larger than and abuts the inverted buckle 33 on the inner side of the housing, so that the activation sleeve 2 and the housing 3 are connected together. Only when the buckle 26 at the tail part of the activation sleeve passes over the transition surface 37 and abuts on the groove 34, the activation sleeve 2 can expand outward and the needle body 1 can be activated. In order to smoothly activate the needle body 1, the guide port 24 is arranged at the rear of the activation sleeve, each of both sides of the tail part of the guide port 24 is provided with the bevel 28, and the inner side of the housing is provided with the guide table 36. In this arrangement, the activation sleeve 2 can expand outward smoothly.

The outer side of the needle cap 11 is slidably fitted with the inner hole 23 of the activation sleeve to prevent the needle cap from displacing and being crooked. The spring 5 is slidably fitted with the guide table 36 of the housing to restrict the radial movement of the spring, thereby reducing the loss of the spring.

The needle cap 11 is provided with the self-locking protrusion 12. The upper part of the self-locking protrusion 12 is provided with the unlocking arm 22 that is located on the activation sleeve 2. The self-locking protrusion 12 and the unlocking arm 22 are fitted with each other to prevent the needle body 1 from being unintentionally activated. The limiting steps 13, the latching steps 14, the guide port 24, and the guide groove 31 are located in the same direction to constitute an axial rail for the needle body 1, so as to prevent the needle core 4 in the needle body 1 from shaking, thereby reducing the pain of the user. The limiting steps 13 of the needle body 1 are fitted with the upper plane of the unlocking arm 22 to control the penetration depth of the needle core 4 in the needle body 1.

Assembly Process:

Firstly, an end of the spring 5 is sleeved on the tail part 17 of the needle body 1, until the end of the spring 5 passes over the protrusion 18 and is hooked to the protrusion 18.

Then, the limiting steps 13 on both sides of the needle body 1 are mounted along the guide port 24 on both sides of the activation sleeve 2. At this moment, the other end of the spring 5 is sleeved on the truncated cone of the housing to effectively prevent the displacement of the spring during activation.

After that, the guide rail 27 of the activation sleeve 2 is mounted along the slide groove 32 of the housing 3, until the buckle 26 of the activation sleeve 2 passes over the inverted buckle 33 of the housing 3.

Finally, the needle cap 11 is mounted along the inner circular hole 61 and the inner flat hole 62 of the protective cap 6, until the fixing buckle 15 is snap-fitted into the through hole 63, so that the needle cap 11 is combined with the protective cap 6.

Working Principle:

The protective cap 6 is rotated to drive the needle cap 11 to be separated from the needle body 1. At this moment, the front end of the activation sleeve 2 is pressed to enable the spring 5 to enter a pre-compressed state. The bevel 28 of the activation sleeve 2 is gradually expanded by the inclined plane on the top of the guide table 36 of the housing 3. At this moment, the latching steps 14 on the needle body 1 can pass through the clamping position 25 of the activation sleeve. Since the spring 5 is in compression, the needle body 1 moves axially along the activation sleeve 2 under the elastic action of the spring 5. The spring 5 then enters a stretched state due to the inertia effect, until the limiting steps 13 on the needle body 1 contact the upper plane of the unlocking arm 22 of the activation sleeve to restrict the needle body 1 from continuing to move axially toward the opening of the housing 3, so that the spring 5 is stopped from stretching to control the penetration depth of the needle core 4. At this moment, the needle core 4 inside the needle body 1 is exposed from the front end of the activation sleeve, and the spring 5 loses its energy gained by compression and then enters a retracting state caused by stretching. After the spring 5 returns to its free length, the needle body 1 completely retracts into the activation sleeve 2, and the needle core 4 inside the needle body 1 is also completely in the activation sleeve.

### Embodiment 2

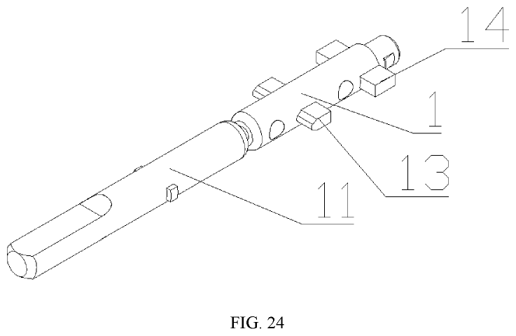

As shown in FIGS. 23 and 24, the miniature safety blood lancet without a cap for minimizing pain in the present embodiment and the miniature safety blood lancet with a protective cap for minimizing pain in embodiment 1 have the same internal structure, and differ in that the front end of the needle cap 11 is not provided with the protective cap 6, namely the miniature safety blood lancet without a cap for minimizing pain in the present embodiment includes the needle body 1, the activation sleeve 2, the housing 3 and the spring 5. The front end of the needle body 1 is provided with the needle cap 11, and the needle cap 11 is not provided with the through hole 16 and the fixing buckle 15. In this arrangement, the miniature safety blood lancet without a cap for minimizing pain in the present embodiment cuts down the cost and reduces waste, while having the same effectiveness as that of the blood lancet with a protective cap. In use, the needle cap 11 is rotated to separate the main body of the needle body 1 from the needle cap 11. Obviously, the above embodiments of the miniature safety blood lancet for minimizing pain of the present invention are only examples for illustrating the present invention clearly, rather than limiting the implementation of the present invention. Other different forms of changes or variations can also be made on the basis of the above description by those of ordinary skill in the art. It is not possible to provide an exhaustive list of all implementation modes herein, and all noticeable changes or variations derived from the technical solutions of the present invention shall fall within the scope of protection of the present invention.

What is claimed is:

1. A miniature safety blood lancet for minimizing pain, comprising:
a needle body,
an activation sleeve,
a housing, and
a spring;
wherein
the activation sleeve is arranged in the housing;
the needle body is arranged in the activation sleeve;
the housing, the activation sleeve and the needle body are slidably connected;
the housing is provided with an inverted buckle;
the activation sleeve is provided with an inner wall;
an inner side of a middle of the inner wall is provided with an unlocking arm, and an outer side of a bottom of the inner wall is provided with a buckle;
a needle cap is arranged at a front end of the needle body;
the needle cap is provided with a self-locking protrusion;
the unlocking arm of the activation sleeve is snap-fitted with the self-locking protrusion of the needle cap and the inverted buckle of the housing, respectively, to fix a positional relationship between the needle body, the activation sleeve and the housing;
the spring is arranged between a bottom of the needle body and the housing;
a guide table is arranged at a bottom of the housing;
the activation sleeve is provided with a bevel;
a top of the guide table abuts an inner side of the bevel; and
when the activation sleeve is pressed, the top of the guide table expands the inner wall towards both sides, and the needle body moves axially to pass through a clamping position of the inner wall under a force of the spring.

2. The miniature safety blood lancet according to claim 1, wherein
both sides of the needle body are provided with limiting steps and latching steps;
a rear of the activation sleeve is provided with a guide port matched with the limiting steps and the latching steps;
a guide groove matched with the limiting steps and the latching steps is arranged within the housing; and
the limiting steps, the latching steps, the guide port, and the guide groove are located in one direction.

3. The miniature safety blood lancet according to claim 2, wherein
the unlocking arm is provided with an upper plane matched with the limiting steps, and
the upper plane of the unlocking arm restricts the needle body from continuing to move axially to control a penetration depth of the needle body.

4. The miniature safety blood lancet according to claim 1, wherein
a slide groove is arranged within the housing, and an outer side of the inner wall is provided with a guide rail matched with the slide groove.

5. The miniature safety blood lancet according to claim 1, wherein
a needle core is pre-embedded in the needle body.

6. The miniature safety blood lancet according to claim 1, wherein
the guide table is arranged on a bottom side wall of the housing, and the guide table abuts both sides of the spring to guide a direction and restrict a lateral movement of the spring, wherein the spring is stretched and compressed in the direction.

7. The miniature safety blood lancet according to claim 1, wherein
a truncated cone is arranged at the bottom of the housing;
a protrusion is arranged on a bottom circumferential wall of the needle body;
a first end of the spring is sleeved on the bottom of the needle body and the first end of the spring passes over the protrusion; and
a second end of the spring is sleeved on the truncated cone of the housing.

8. The miniature safety blood lancet according to claim 1, further comprising a protective cap; wherein
the protective cap is sleeved on the needle cap, and the protective cap is snap-fitted with the needle cap.

9. The miniature safety blood lancet according to claim 2, further comprising a protective cap; wherein
the protective cap is sleeved on the needle cap, and the protective cap is snap-fitted with the needle cap.

10. The miniature safety blood lancet according to claim 3, further comprising a protective cap; wherein
the protective cap is sleeved on the needle cap, and the protective cap is snap-fitted with the needle cap.

11. The miniature safety blood lancet according to claim 4, further comprising a protective cap; wherein
the protective cap is sleeved on the needle cap, and the protective cap is snap-fitted with the needle cap.

12. The miniature safety blood lancet according to claim 5, further comprising a protective cap; wherein
the protective cap is sleeved on the needle cap, and the protective cap is snap-fitted with the needle cap.

13. The miniature safety blood lancet according to claim 6, further comprising a protective cap; wherein
the protective cap is sleeved on the needle cap, and the protective cap is snap-fitted with the needle cap.

14. The miniature safety blood lancet according to claim 7, further comprising a protective cap; wherein
the protective cap is sleeved on the needle cap, and the protective cap is snap-fitted with the needle cap.
